(19) United States
(12) Patent Application Publication
(10) Pub. No.: US 2025/0249177 A1
(43) Pub. Date: Aug. 7, 2025

(54) Device for a controlled ejection of a liquid solution

(71) Applicants: Centre national de la recherche scientifique, Paris (FR); UNIVERSITE DE HAUTE ALSACE, Mulhouse Cedex (FR)

(72) Inventor: Valeriy LUCHNIKOV, Mulhouse Cedex 2 (FR)

(21) Appl. No.: 18/856,317

(22) PCT Filed: Apr. 13, 2023
(86) PCT No.: PCT/EP2023/059686

§ 371 Date: Oct. 11, 2024

(30) Foreign Application Priority Data
Apr. 13, 2022 (EP) 22305535.1

(51) Int. Cl. A61M 5/31; A61M 5/19; A61M 5/315

(52) U.S. Cl. CPC A61M 5/3148; A61M 5/19; A61M 5/3129; A61M 5/31513; A61M 2005/3115; A61M 2005/3123; A61M 2005/3131

(57) ABSTRACT

The invention concerns a device (10) comprising: — a barrel (12); — an elastic system (14) separating the barrel into a solution chamber (36) and a depressurized chamber (38); — a first opening (16) to the solution chamber; — a piston (18) able to be moved inside the depressurized chamber from a rest configuration to a pulled configuration in order to suck said solution into the solution chamber; — a second opening (20) communicating with the depressurized chamber via a flow restrictor (21), and — a regulation system (22) able to close the second opening to make the depressurized chamber airtight when the piston is sucking the solution and able to open the second opening to release the elastic system and to eject the solution out of the solution chamber.

### FIELD OF THE INVENTION

The present invention concerns a device for a controlled ejection of a liquid solution. In particular, the liquid solution is a medical solution or a food solution.

The invention also concerns a method of loading the liquid solution into such a device.

### BACKGROUND OF THE INVENTION

Many medical solutions require a slow mode of administration, in order to avoid undesirable and/or acute effects and to obtain the best therapeutic effect.

Slow injection with the use of traditional syringes requires special training for doctors and nurses, but is still a tedious and time-consuming operation, especially if large amounts of medication are to be administered to a patient. In addition, the rate of administration can vary from patient to patient.

Technical solutions allowing this type of administration are available but they require an external energy supply (electrical or mechanical) or a permanent and constraining human intervention. Moreover, they do not meet all the technical and financial requirements for use by the medical profession.

In particular, syringe and infusion pumps are efficient but not completely appropriate for mobile medical interventions because of their energy dependency and their price makes them not affordable devices. On the other side, elastomeric pumps and drip sets with bag may be considered as more mobile solutions but imply additional devices to operate, which is not suitable for emergency medical interventions. For example, the document WO 2002/22189 discloses such an elastomeric pump.

### SUMMARY OF THE INVENTION

One of the aims of the invention is to propose a device for injecting a liquid solution, as a medical solution for example, at a slow and controlled flowrate, while remaining cost efficient.

For this purpose, the invention relates to a device for a controlled ejection of a liquid solution comprising:
a barrel defining an internal volume;
an elastic system separating the internal volume into a solution chamber and a depressurized chamber;
a first opening configured to allow the inlet and the outlet of said solution into the solution chamber;
a piston able to be moved inside the depressurized chamber from a rest configuration to a pulled configuration in order to suck said solution into the solution chamber through the first opening by the deformation of the elastic system creating a vacuum into the solution chamber;
a second opening communicating with the depressurized chamber via a flow restrictor, and
a regulation system able to close the second opening to make the depressurized chamber airtight when the piston is sucking the solution and able to open the second opening to allow an airflow to flow inside the depressurized chamber via the flow restrictor to release the elastic system and to eject the solution out of the solution chamber through the first opening.

Thanks to these features, the elastic system and the piston enable the suction of the solution inside the device through vacuum suction. The flow restrictor enables to control the airflow inlet inside the device and in consequence the flow rate of the solution ejection out of the device. The invention makes the work of the user, and notably a medical staff, more comfortable and less stressful, and limits the rate of administration to a safe level, without the need for expensive and complex electronic devices.

The device according to the invention comprises one or more of the following features, taken solely, or according to any technically feasible combination:
the elastic system is an elastic membrane fixed relatively to the barrel, the elastic membrane being advantageously made of polyisoprene, silicone or latex;
the elastic system is a mobile shutter in the internal volume of the barrel, the mobile shutter being connected to a spring arranged inside the depressurized chamber or inside the solution chamber;
the device comprises a cap removably cooperating with the barrel and defining the first opening;
the device comprises position-holding means configured to keep the piston in the pulled configuration;
the position-holding means are a stop protruding from the barrel inside the internal volume, the stop being able to cooperate with positioners to maintain the piston in the pulled configuration;
the device comprises a handle for moving the piston from the rest configuration to the pulled configuration;
the regulation system comprises a screw able to move in a matching hole from a screwed-in configuration blocking the fluidic communication between the second opening and the flow restrictor, to an unscrewed configuration enabling the fluidic communication between the second opening and the flow restrictor;
the flow restrictor is a capillary system extending between the second opening and the depressurized chamber;
the capillary system comprises at least one capillary channel extending between the second opening and the depressurized chamber;
the screw comprises externals threads and the matching hole comprises internal threads, the capillary system being a spiral capillary channel defined between the external threads and the internal threads.
the flow restrictor is a porous track-etched membrane;
the piston is able to move from the rest configuration to the pulled configuration through a translation movement, without rotation; and
the device comprises a screw plunger cooperating with the piston, the piston being able to move from the rest configuration to the pulled configuration through a translation and rotation movement;
the device further comprises a rod, the rod comprising the flow restrictor, the piston being connected to the rod;
the regulation system comprises:
   a screw cap mounted movable onto the rod between a fully screwed on configuration and a plurality of unscrewed configurations, the second opening being defined by the screw cap; and
   a porous plug arranged between the second opening and the flow restrictor;
   wherein the screw cap pushes the porous plug against the rod so that the porous plug is compressed to make the depressurized chamber airtight when the screw cap is in the fully screwed on configuration, wherein the screw cap enables the porous plug to expand, allowing air to flow inside the depressurized chamber via the flow restrictor to release the elastic membrane when the screw cap is in an unscrewed configuration;
the regulation system further comprises at least one sealing member, the at least one sealing member being configured to direct the flow of air from the second opening to the flow restrictor through the porous plug in an airtight manner;
the position-holding means are configured to keep the piston in position along the longitudinal axis, the position-holding means comprising several groups of stops protruding radially from the rod towards the barrel, the position-holding means comprising a stop protruding from the barrel inside the internal volume, the rod being mounted rotatable around the longitudinal axis between an unlocked configuration and a locked configuration, the stops protruding radially from the rod being away from the stops protruding from the barrel when the rod is in the unlocked configuration, the stops protruding radially from the rod and the stops protruding from the barrel cooperating so that the piston is kept in position along the longitudinal axis when the rod is in the locked configuration;
the elastic system comprises a support, the support being fixedly mounted into the internal volume of the barrel, the elastic membrane being mounted onto the support;
the device further comprises an airflow adjustment system comprising at least one through-hole extending in the barrel, the at least one through-hole being able to put in fluidic communication the depressurized chamber with ambient air, the at least one through-hole being arranged so as to be plugged by the elastic system when the piston is in a pulled configuration and opened, when the piston is in the rest configuration.

The invention also concerns a device for a controlled ejection of a liquid solution comprising:
a barrel defining an internal volume;
an elastic system separating the internal volume into a solution chamber and a depressurized chamber;
a first opening configured to allow the inlet and the outlet of said solution into the solution chamber;
a piston able to be moved inside the depressurized chamber from a rest configuration to a pulled configuration in order to suck said solution into the solution chamber through the first opening by the displacement of the elastic system creating a vacuum into the solution chamber, the elastic system following the movement of the piston, the elastic system having elastic forces, which tends it to come back in a rest configuration;
a second opening communicating with the depressurized chamber via a flow restrictor, and
a regulation system able to close the second opening to make the depressurized chamber airtight when the piston is sucking the solution and able to open the second opening to allow an airflow to flow inside the depressurized chamber via the flow restrictor to release the elastic system and to eject the solution out of the solution chamber through the first opening.

The device according to the invention comprises one or more of the above detailed features, taken solely, or according to any technical feasible combination.

The invention also concerns a method of loading a liquid solution into a device the method comprising at least the following steps:
providing a reservoir containing the solution,
providing the device as defined above, the piston being in the rest configuration and the regulation system closing the second opening,
placing the first opening in fluidic contact with solution contained in the reservoir,
moving the piston from the rest configuration to the pulled configuration and sucking said solution into the solution chamber.

The invention also concerns a method of delivering a liquid solution loaded into such a device, the method comprising at least the following steps:
providing the device as defined above, the device being loaded with said solution in the solution chamber, the piston being in the pulled configuration and the regulation system closing the second opening,
operating the regulation system to open the second opening allowing an airflow to flow inside the depressurized chamber via the flow restrictor to release the elastic system,
ejection of the solution out of the solution chamber through the first opening.

### BRIEF DESCRIPTION OF THE DRAWINGS

The invention and its advantages will be better understood upon reading the following description, which is given solely by way of non-limiting example and which is made with reference to the appended drawings, in which:

FIG. 1 is a cross sectional view of a first embodiment of a device according to the invention in an assembled configuration;

FIG. 2 is a cross sectional view of the device of FIG. 1 in a disassembled configuration;

FIG. 3 is a cross sectional view of the device of FIG. 1 during the loading of the liquid solution;

FIG. 4 is a cross sectional view of the device of FIG. 1 during the delivering of the liquid solution;

FIG. 5 is a cross sectional view of a second embodiment of a device according to the invention;

FIG. 6 is a cross sectional view of the device of FIG. 5 during the delivering of the liquid solution;

FIG. 7 is a cross sectional view of a third embodiment of a device according to the invention,

FIG. 8 is a cross sectional view of a fourth embodiment of a device according to the invention,

FIG. 9 is a cross sectional view of a fifth embodiment of a device according to the invention,

FIG. 10 is a cross sectional view of a sixth embodiment of a device according to the invention,

FIG. 11 is a cross sectional view of a seventh embodiment of a device according to the invention;

FIG. 12 is a cross sectional view of an example of a device according to the invention;

FIG. 13 is a cross sectional view along a plane XIII-XIII substantially perpendicular to the longitudinal axis X of the device of FIG. 12;

FIG. 14 is a cross sectional view similar to the view of FIG. 13, of a variant of the device of FIG. 12;

FIG. 15 is a cross sectional view similar to the view of FIG. 14, of another variant of the device of FIG. 12;

FIG. 16 is a cross sectional view of another example of a device according to the invention;

FIG. 17 is a cross sectional view of another example of a device according to the invention, in which the elastic system plugs both through-holes;

FIG. 18 is a cross sectional view of the device of FIG. 17, in which the elastic system plugs only one through-hole, the other through-hole being opened.

### DESCRIPTION OF A FIRST EMBODIMENT OF THE INVENTION

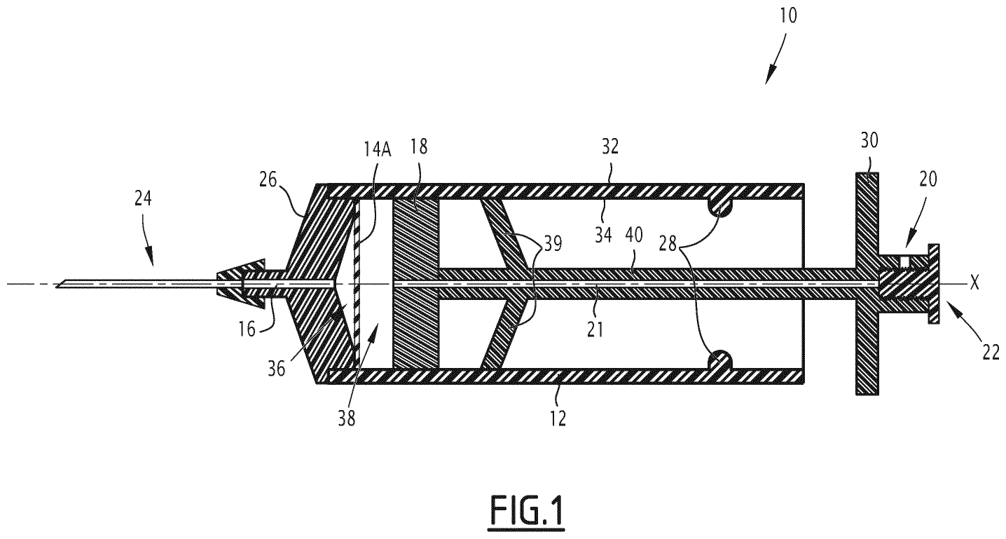

A device 10 for a controlled ejection of a liquid solution is represented on FIG. 1.

The liquid solution is a liquid fluid, which may comprise solid particles floating in the fluid.

The liquid solution is notably a medical solution, in particular a medical solution with slow administration rates.

For example, intravenous injections of Tramamdol should be given slowly over 2 to 3 minutes.

As another example, each dose of Digoxin should be given by intravenous infusion over 10 to 20 minutes. Rapid intravenous injection may cause vasoconstriction producing hypertension and/or decreased coronary flow. A slow injection rate is therefore important in hypertensive heart failure and acute myocardial infarction.

As another example, adrenaline injection or infusion is the drug of choice for the first-line treatment of cardiopulmonary arrest, anaphylactic shock, and certain severe shock states (in resuscitation). For example, in case of a cardiac arrest, an intravenous injection of 0.5 μg during 5 minutes is carried out. As another example, in case of cardiac arrest, an intravenous infusion with a rate comprised between 2 μg per minute to 10 μg per minute is carried out.

The device may be used for medicine administration by injection route for emergency care or humanitarian applications, for wildlife veterinarian applications and/or for the patient transportation in isolated environment.

The injection or infusion with the device may be intravenous, intramuscular, nasogastric or subcutaneous.

In a variant, the liquid solution may be a food solution. In particular, liquid nutrients should be delivered slowly into the gastrointestinal tract, for example at a rate lower than 30 ml/min, to avoid adverse effects such as abdominal discomfort, nausea, satiety and cramping.

In a variant, the liquid solution may be a glue, a silicone or a painting.

Referring to FIG. 1, the device 10 extends along a longitudinal axis X.

The device 10 comprises a barrel 12, an elastic system 14, a first opening 16, a piston 18, a second opening 20, an air flow restrictor 21 and a regulation system 22.

The device 10 may further comprise a needle 24, a cap 26, position-holding means 28 and a handle 30.

The barrel 12 presents a cylindrical shape, extending along the longitudinal axis X. The transversal section of the barrel 12 is notably circular. In a variant, the transversal section may be a square or a hexagonal for example. The barrel 12 extends advantageously along a length comprised between 5 cm and 50 cm along the longitudinal axis X. The barrel 12 defines an external surface 32 and an internal surface 34. The barrel 12 defines an internal volume.

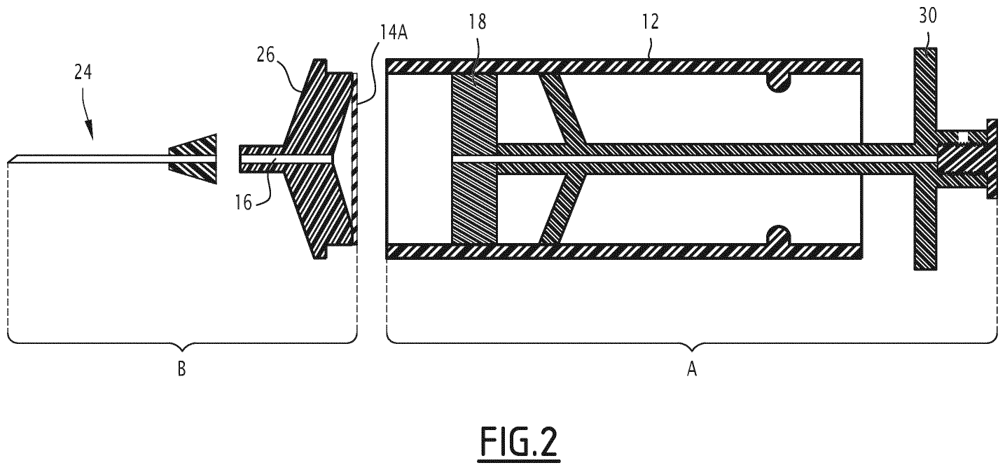

The cap 26 is arranged at an extremity of the barrel 12 along the longitudinal axis X. The cap 26 is advantageously made of plastic, for example polypropylene. The cap 26 is removably cooperating with the barrel 12. In other words, the cap 26 may be fixed to the barrel 12 in an assembled configuration of the device 10. The cap 26 may be easily removed from the barrel 12 by a user in a dissembled configuration of the device 10, represented on FIG. 2.

Referring to FIG. 2, the device 10 comprises a reusable part A composed notably of the barrel and the piston, and a disposable part B composed of the membrane 14A, the cap 26 and the needle 24. This enables to decrease the price of the medical device considering multiple uses. Moreover, the healthcare and the sterilization are carried out easily.

The cap 26 defines the first opening 16. The first opening 16 is notably defined at the center of the cap 26. In an alternative, the first opening 16 is off-center. The first opening 16 is a channel extending along the longitudinal axis X in the cap 26.

The elastic system 14 is here an elastic membrane 14A fixed relatively to the barrel 12. The elastic membrane 14A may be fixed to the internal surface 34 of the barrel 12. In a variant, the elastic membrane 14A is fixed to the cap 26, as represented on FIG. 2, and when the device is assembled the elastic membrane 14A is thus fixed relatively to the barrel 12. The elastic membrane 14A is advantageously made of polyisoprene, silicone or latex. Optionally, the elastic membrane 14A presents a specific color. Said specific color makes it easier to read the volume of solution loaded into a solution chamber 36 of the internal volume, to control the absence of bubbles in the loaded solution and to control the conformity of the injection.

As visible on the Figures, the elastic system 14 separates the internal volume into the solution chamber 36 and a depressurized chamber 38. In other words, there is no fluidic communication between the solution chamber 36 and the depressurized chamber 38, due to the elastic system 14.

The first opening 16 is configured to allow the inlet and the outlet of the solution into the solution chamber 36.

The needle 24 is arranged in the extension of the first opening 16. The needle 24 is fixed to the cap 26. In alternative, the device 10 is devoid of a needle.

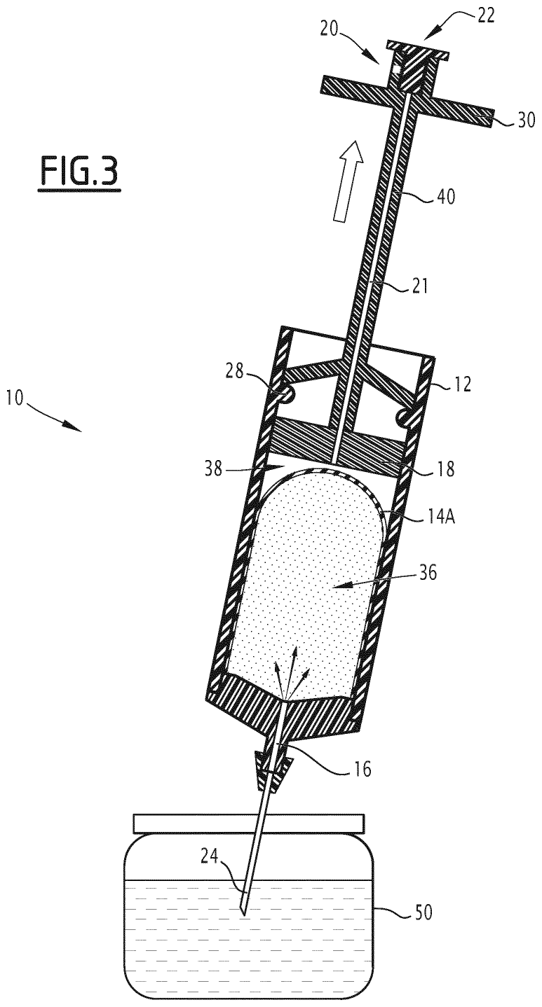

The piston 18 is able to be moved inside the depressurized chamber 38, by translation along the longitudinal axis X. Here, the piston 18 is able to move through a translation movement along the longitudinal axis X, without rotation. However, the skilled person will understand that the user may move the piston 18 through a translation movement with eventually a rotation movement. The piston 18 is able to move from a rest configuration, represented on FIG. 1, to a pulled configuration, represented on FIG. 3. In particular, when moving from the rest configuration to the pulled configuration, the piston 18 is moving in the opposite direction from the first opening 16. When moving from the rest configuration to the pulled configuration, the elastic membrane 14A is deformed. Indeed, the volume of the depressurized chamber 38 between the elastic membrane 14A and the piston 18 is airtight and the elastic membrane 14A therefore follows the movement of the piston 18 to maintain the balance between the atmospheric pressure, the pressure in the depressurized chamber 38 and the tension of the elastic membrane 14A. This deformation of the elastic membrane 14A creates a vacuum into the solution chamber 36. In consequence, this vacuum sucks the solution from the outside of the device 10 into the solution chamber 36 part through the first opening 16, as illustrated on FIG. 3.

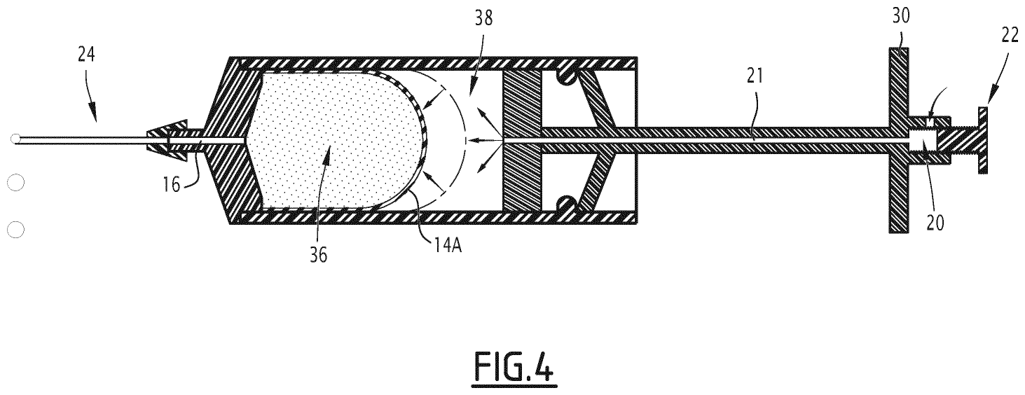

The position-holding means 28 are configured to keep the piston 18 in the pulled configuration. As visible in the FIG. 1, the position-holding means 28 are here a stop 69 protruding from the internal surface 34 of the barrel 12 inside the internal volume. The stop 69 is in particular protruding circumferentially. The stop 69 is able to cooperate with the piston 18, and in particular with positioners 39 to maintain the piston 18 in the pulled configuration, as visible on FIGS. 3 and 4.

The handle 30 is configured to move the piston 18 from the rest configuration to the pulled configuration. The handle 30 is connected to the piston 18 through a rod 40 extending along the longitudinal axis X. In particular, the handle 30 is designed to be grabbed by the hand of a user. By pulling the handle 30, the piston 18 is pulled from the rest configuration to the pulled configuration

The second opening 20 communicates with the depressurized chamber 38 via the air flow restrictor 21. The flow restrictor 21 is here a capillary system extending between the second opening 20 and the depressurized chamber 38. The capillary system comprises at least one capillary channel extending along the longitudinal axis X between the second opening 20 and the depressurized chamber 38. The capillary channel(s) extend(s) inside the rod 40 and the piston 18. On the example represented on FIG. 1, the capillary system comprises here a single capillary channel. In a variant, not shown, the capillary system comprises advantageously several capillary channels extending in parallel. Each capillary channel is thin enough to allow capillary action to take place. In particular, each capillary channel presents advantageously a diameter comprised between 0.05 mm to 1 mm.

As represented on FIG. 3, the regulation system 22 is able to close the second opening 20 to make the vacuum chamber 28 airtight when the piston 18 is drawing the solution. As represented on FIG. 4, the regulation system 22 is further able to open the second opening 20 to allow an airflow to flow inside the vacuum chamber 28 via the air flow restrictor 21 to release the elastic membrane 14A and to eject the solution out of the solution chamber 36 through the first opening 16.

The regulation system 22 comprises here a screw able to move in a matching hole from a screwed-in configuration blocking the fluidic communication between the second opening 20 and the flow restrictor 21, as represented on FIGS. 1 to 3, to an unscrewed configuration enabling the fluidic communication between the second opening 20 and the flow restrictor 21, as represented on FIG. 4.

The solution flowrate out of the first opening 16 is linked to the flowrate of the air entering the depressurized chamber 38 through the flow restrictor 21. This air flowrate Q_{AIR} is defined by the aerodynamic resistance in the capillary channel given by the Hagen-Poiseuille equation:

$${Q}_{AIR} = ( π {r}^{4} / 8 η l ) Δ P$$

with viscosity η, capillary channel length l, capillary channel radius r, and pressure difference between the atmospheric pressure and the pressure of the depressurized chamber 38 ΔP=P_{ATM}−P_{VAC.CHAMB}.

The high influence of r⁴ term of aerodynamic resistance on capillary allows the control of the administration flowrate within a wide range of possibilities. Moreover, the skilled person will understand that by using N capillaries with different geometrical characteristics (length l, radius r), and thus respectively different aerodynamic resistance, the device 10 may deliver the liquid solution within a range of 2^{N} configurations.

### OPERATION OF THE INVENTION

The operation of the device 10 will now be described.

The operation comprises a method of loading a liquid solution into the device 10, in reference to FIG. 3.

A reservoir 50 containing the solution is provided. A device 10 as described above is also provided, the piston 18 being in the rest configuration and the regulation system 22 closing the second opening 20, as represented on FIG. 1.

The first opening is placed in fluidic contact with the solution contained in the reservoir 50. In particular, the needle 24 is placed inside the reservoir 50. In the alternative in which the device 10 is devoid of a needle, the cap 26 and the first opening 16 are directly placed in contact with the solution contained in the reservoir 50.

Then, the user grabs the handle 30 and pulls it along the longitudinal axis X, as illustrated by the arrow represented on FIG. 3. The translation movement of the handle 30 leads to the piston 18 to move from the rest configuration to the pulled configuration.

A depressurization is created in the depressurized chamber 38 whose pressure becomes lower than the atmospheric pressure. The elastomeric membrane 14A then expands because of this pressure difference, and the solution is sucked through the needle 24 and the first opening 16 to the solution chamber 36.

When the piston 18 is pulled enough to be locked in position in the barrel 12 thanks to the contact between the position-holding means 28 and the positioners 39, the pulled configuration is maintained due to the suction from the depressurized chamber 38 on the elastic membrane 14A which is now maintained in pulled configuration even if its elastic forces tend it to come back in rest configuration.

In alternative to the method of loading a liquid solution into the device 10 carried out by the user, the device 10 may be loaded upstream, for example in a factory, and sold directly loaded and in the pulled configuration to the user.

Then, the operation comprises a method of delivering a liquid solution, in reference to FIG. 4.

The device 10 is connected to the patient body through the needle 24 directly inserted to the patient body, or alternatively using a medical tubing between the device 10 and the needle 24 or between the device 10 and a catheter.

As visible on FIG. 4, the regulation system 22 is operated to open the second opening 20 allowing an airflow to flow inside the depressurized chamber 38 via the flow restrictor to release the elastic membrane 14A.

In particular, the screw is unscrewed, allowing the air to flow through the second opening 20, through the flow restrictor 21 and finally into the depressurized chamber 38 which implies the balance of the atmospheric pressure, the pressure exerted by the elastic membrane 14A on the solution, and the pressure in the depressurized chamber 38. It implies also the deflation of the solution chamber 36 under mechanical forces applied by the elastic membrane 14A on the solution.

In consequence, the solution is then pushed outside of the device 10 through the first opening 16 and the needle 24, and delivered for example to the patient.

The deliverance of the solution may be stopped at any time by closing the regulation system 22.

### Description of a Second Embodiment of the Invention

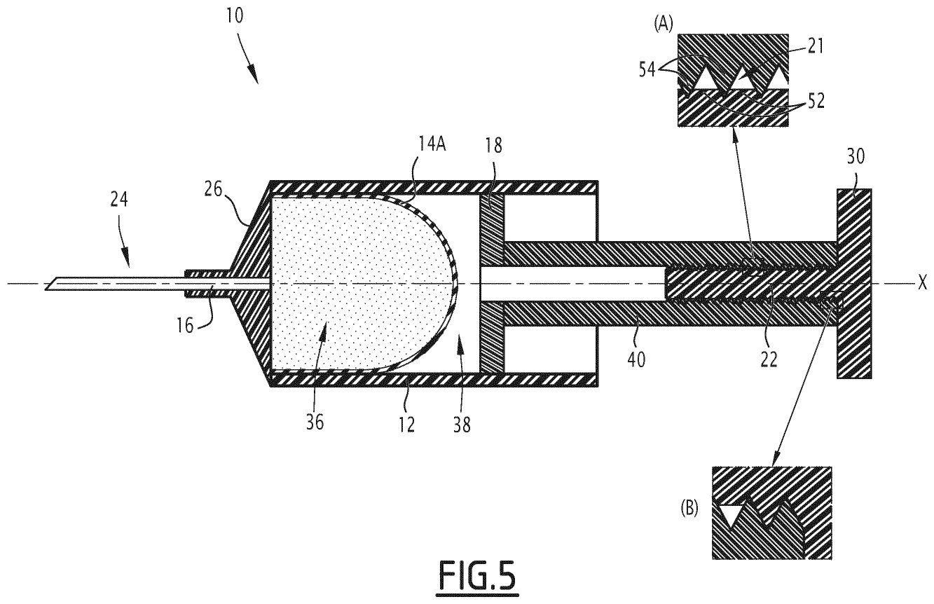

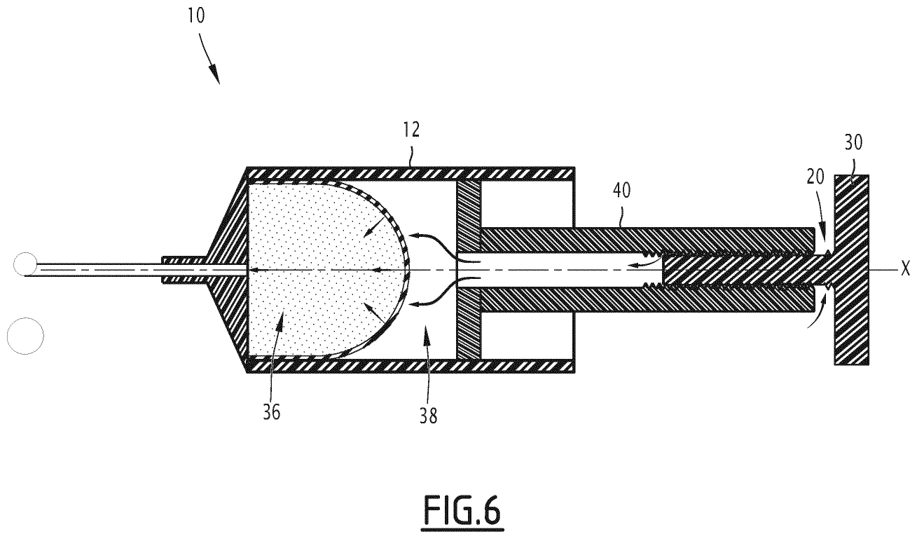

A second embodiment of the device 10 according to the invention will be described below, referring to FIGS. 5 and 6.

The device 10 according to the second embodiment is similar to device 10 according to the first embodiment explained above except the features described below.

In particular, the regulation system 22 comprises here a screw cooperating with a matching hole arranged in the rod 40. The screw comprises externals threads 52 and the matching hole comprises internal threads 54, as visible on the enlargement (A) of FIG. 5.

As visible on FIGS. 5 and 6, the capillary system 21 is here a spiral capillary channel defined between the external threads 52 and the internal threads 54. The internal threads 54 are triangular. The external threads 52 are trapezoidal except the last threads arranged next to the handle 30, which are triangular, as visible on the enlargement (B).

In a variant, the external and the internal threads 52, 54 may be rectangular, so that the air capillary has a rectangular cross-section.

The regulation system 22 comprises here the screw able to move in the matching hole from a screwed-in configuration blocking the end of the spiral capillary channel, as visible on the enlargement (B) of FIG. 5, to an unscrewed configuration opening the spiral capillary channel and enabling the air communication between the second opening 20 and the flow restrictor 21, as represented on FIG. 6.

The piston 18, the rod 40 and the screw may be manufactured using standard methods of making internal and external threads.

The regulation system 22 enables to adjust the delivery rate of the solution. Indeed, as the aerodynamic resistance of the capillary is proportional to its length, the delivery rate may be adjusted within by the number of screw turns.

In particular, in case of the rectangular threads, the formula for the aerodynamic resistance of the spiral capillary channel reads as follows:

$$R \approx \frac{12}{- 0.63 ( h / w )} \frac{η \sqrt{{( 2 π r )}^{2} + {a}^{2}}}{{h}^{3} w} ( N - n )$$

The flow rate Q of the air in the device 10 is:

$$Q = \frac{Δ P}{R} = \frac{1 - 0.63 ( h / w )}{12} \frac{{h}^{3} w}{η \sqrt{{( 2 π r )}^{2} + {a}^{2}}} \frac{Δ P}{N - n}$$

where h,w are the dimensions of the cross-sectional area, η is the viscosity of the air, L is the length of the capillary, N is the number of turns required to completely separate the screw from the device, n is the number of turns made to open the capillary.

As visible on the FIGS. 5 and 6, the device 10 according to the second embodiment is devoid of position-holding means 28. The friction coefficient between the piston 18 and the barrel 12 is here sufficiently high to maintain the piston 18 in position when the solution chamber 56 is loaded, and the elastic membrane is inflated.

The operation of the second embodiment is similar to the operation of the first embodiment and will not be described again.

### Description of a Third Embodiment of the Invention

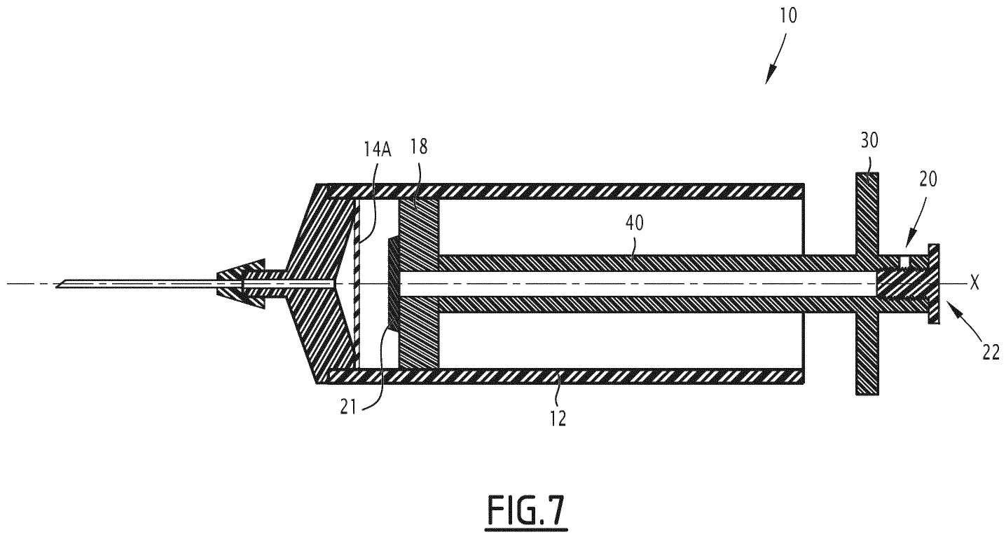

A third embodiment of the device 10 according to the invention will be described below, referring to FIG. 7.

The device 10 according to the third embodiment is similar to device according to the first embodiment explained above except the features described below.

The air flow restrictor 21 is here a porous track-etched membrane. A track-etched membrane is a porous system comprising a thin polymer foil with channels crossing the membrane.

The porous track-etched membrane is arranged in the depressurized chamber 38 and covers the extremity of the channel arranged inside the rod 40 between the depressurized chamber 38 and the second opening 20.

The porous track-etched membrane is produced by irradiation of a polymer membrane by ions, accelerated to the energy of a few MeV in an industrial particle accelerator, followed by chemical etch if the ion tracks in the polymer. A broad spectrum of porous track-etched membranes exists with different permeabilities to air. For example, a 25 μm thick polycarbonate films, with a pore size of 0.05 μm, a porosity of 1.2%, is associated to an air flow rate of the order of 20 mL.min⁻¹.cm⁻² at a pressure difference of 0.7 bar, while at the pore size of 0.05 μm and a porosity 4.7% and with a same pressure difference, the air flow rate increases to approximatively 200 mL.min⁻¹.cm⁻².

As visible on the FIG. 7, the device 10 according to the third embodiment is devoid of position-holding means 28, in a similar way to the second embodiment.

The operation of the third embodiment is similar to the operation of the first embodiment and will not be described again.

### Description of a Fourth Embodiment of the Invention

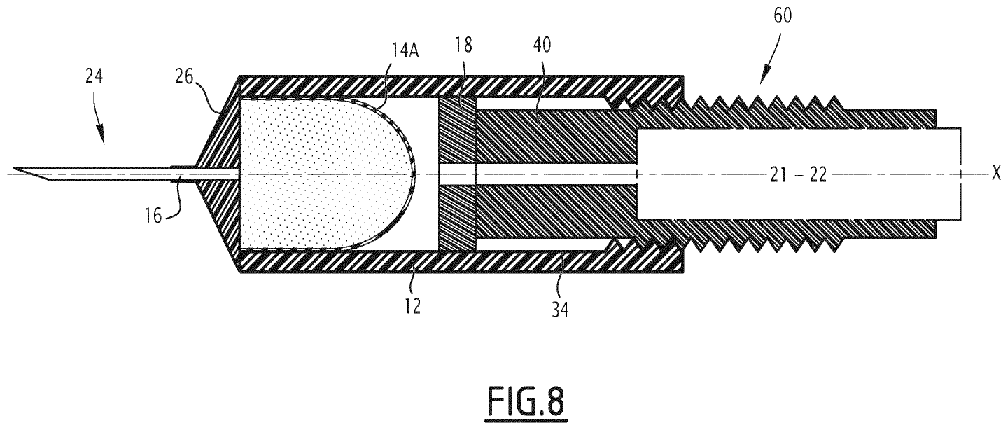

A fourth embodiment of the device 10 according to the invention will be described below, referring to FIG. 8.

The device 10 according to the fourth embodiment is similar to device according to the first embodiment explained above except the features described below.

The device 10 comprises a screw plunger 60 configured to move the piston 18 along the longitudinal axis X.

To that effect, the screw plunger 60 is cooperating with the piston 18, through the rod 40. The rod 40 comprises external threads cooperating with internal threads arranged on the internal surface 34 of the barrel 12.

By rotation the screw plunger 60, the screw plunger 60 is able to move the piston 18 from the rest configuration to the pulled configuration. Here, the piston 18 is able to move from the rest configuration to the pulled configuration through a translation movement and a rotation movement along the longitudinal axis X.

The screw plunger 60 acts as position-holding means.

The flow restrictor 21 and the regulation system 22 are shown on FIG. 8 schematically by a box. The flow restrictor 21 and the regulation system 22 may be implemented as in the first embodiment, second embodiment or third embodiment.

The operation of the fourth embodiment is similar to the operation of the first embodiment and will not be described again.

### Description of a Fifth Embodiment of the Invention

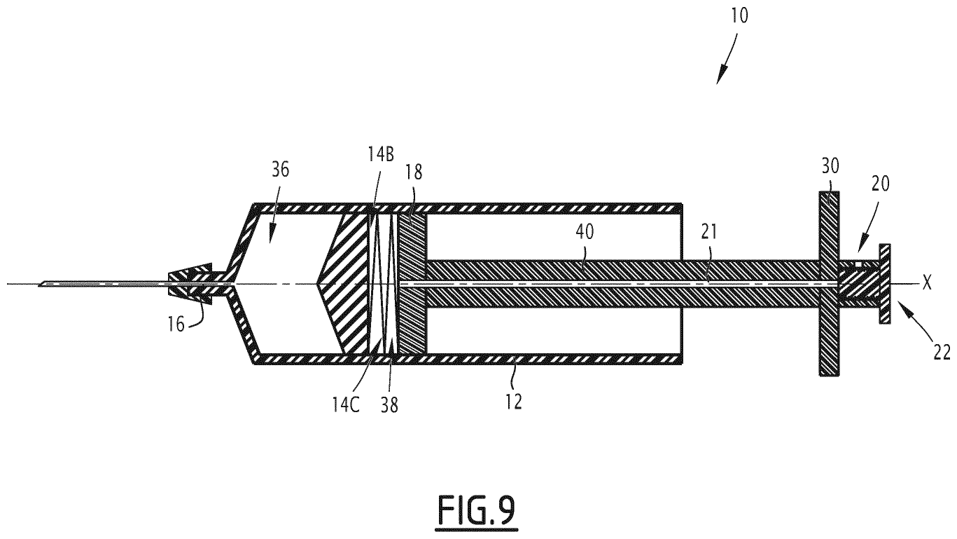

A fifth embodiment of the device 10 according to the invention will be described below, referring to FIG. 9.

The device 10 according to the fifth embodiment is similar to device according to the first embodiment explained above except the features described below.

The elastic system 14 is here a mobile shutter 14B in the internal volume of the barrel 12. The mobile shutter 14B is in particular able to move along the longitudinal axis X between the first opening 16 and the piston 18.

As visible on FIG. 9, the mobile shutter 14B is connected to a spring 14C arranged inside the depressurized chamber 38 between the mobile shutter 14B and the piston 18.

The spring 14C may be a helical spring. In a variant, the spring 14C may be a conical spring allowing a smaller footprint in the depressurized chamber 38.

When moving from the rest configuration to the pulled configuration, the length of the spring is hold equal as the volume of the depressurized chamber 38 between the mobile shutter 14B and the piston 18 is airtight. The mobile shutter 14B therefore follows the movement of the piston 18 to maintain a constant internal volume in the depressurized chamber 38. This movement of the mobile shutter 14B creates a vacuum into the solution chamber 36. In consequence, this vacuum sucks the solution from the outside of the device 10 into the solution chamber 36 part through the first opening 16, as illustrated on FIG. 9.

When the regulating system 22 opens the second opening 20, air flows in the depressurized chamber 38, allowing the spring to relax to its extended state and to push the mobile shutter 14B in the direction of the first opening 16. The shutter pushes then the solution outside the device.

As visible on the FIG. 9, the device 10 according to the fifth embodiment is devoid of position-holding means 28, in a similar way to the second embodiment.

### Description of a Sixth Embodiment of the Invention

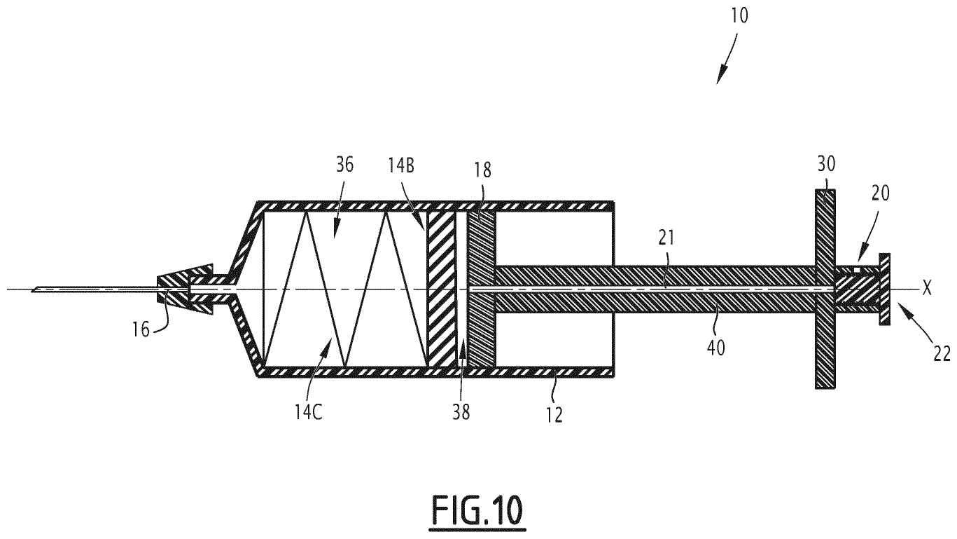

A sixth embodiment of the device 10 according to the invention will be described below, referring to FIG. 10.

As in the fifth embodiment, the elastic system 14 is a mobile shutter 14B in the internal volume of the barrel 12. The mobile shutter 14B is in particular able to move along the longitudinal axis X between the first opening 16 and the piston 18.

As visible on FIG. 10, the mobile shutter 14B is connected to a spring 14C arranged inside the solution chamber 36 between the mobile shutter 14B and the cap 26.

The spring 14C may be a helical spring. In a variant, the spring 14C may be a conical spring allowing a smaller footprint in the depressurized chamber 38.

When moving from the rest configuration to the pulled configuration, vacuum is created in the depressurized chamber 38 between the piston 18 and the shutter 14B. The mobile shutter 14B therefore follows the movement of the piston 18 to maintain a constant internal pressure in the depressurized chamber 38. This movement of the mobile shutter 14B creates a vacuum into the solution chamber 36. In consequence, this vacuum sucks the solution from the outside of the device 10 into the solution chamber 36 part through the first opening 16, as illustrated on FIG. 10.

When the regulating system 22 opens the second opening 20, air flows in the depressurized chamber 38, allowing the spring 14C to relax to its unstrained state and to push the mobile shutter 14B in the direction of the first opening 16. The shutter 14B pushes then the solution outside the device.

As visible on the FIG. 10, the device 10 according to the sixth embodiment is devoid of position-holding means 28, in a similar way to the second embodiment.

### Description of a Seventh Embodiment of the Invention

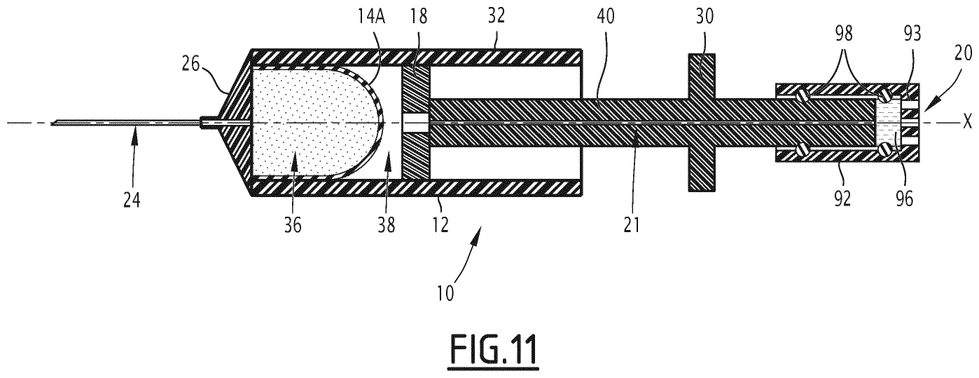

A seventh embodiment of the device 10 according to the invention will be described below, referring to FIG. 11.

The device 10 according to the seventh embodiment is similar to the device according to the first embodiment explained above except for the features described below.

According to the seventh embodiment, the device 10 comprises the rod 40. The rod 40 comprises the flow restrictor 21, in particular the capillary system, notably the at least one capillary channel.

As shown on FIG. 11, the second opening 20 is defined by a screw cap 92 of the regulation system 22. In particular, the second opening 20 is made up of at least one tubular hole 93 extending substantially parallel to the longitudinal axis X when the screw cap 92 is mounted onto the rod 40.

The regulation system 22 comprises the screw cap 92. Advantageously, the regulation system 22 further comprises a porous plug 96. Advantageously, the regulation system 22 further comprises at least one sealing member 98.

The screw cap 92 is configured to cooperate with the rod 40. In particular, the screw cap 92 comprises internal threads (non-illustrated) and the rod 40 comprises external threads (non-illustrated). The internal threads of the screw cap 92 and the external threads of the rod 40 are designed to cooperate to enable screwing and unscrewing of the screw cap 92. The screw cap 92 can be arranged between a fully screwed on configuration and a plurality of unscrewed configurations.

The porous plug 96 is arranged between the second opening 20 and the flow restrictor 21 so that the air flowing from the second opening 20 to the flow restrictor 21 passes through the porous plug 96. For example, the porous plug 96 is configured for filtering the air flowing from the second opening 20 to the flow restrictor 21. In particular, the porous plug 96 comprises pores, the size of which being adapted to let the air flow while stopping the passage of extraneous particles such as dust. Advantageously, the porous plug 96 is made up of a medical foam. Here “medical foam” means a foam that has been treated according to medical standards relating for example to sterilization. The medical foam comprises for example polyethylene and/or polyurethane.

In the fully screwed on configuration, the screw cap 92 pushes the porous plug 96 against the rod 40 so that the porous plug 96 is compressed to make the depressurized chamber 38 airtight. Advantageously, in the compressed state, the porous plug 96 becomes airproof. In the plurality of unscrewed configurations, the screw cap 92 enables the porous plug 96 to expand, allowing air to flow inside the depressurized chamber 38 via the air flow restrictor 21 to release the elastic membrane 14A. The air flow rate through the porous plug 96 depends on the unscrewed configuration in which the screw cap 92 is. The more the screw cap 92 is unscrewed, greater the air flow rate. For example, the air flow rate through the porous plug 96 varies between 0 mL/min and 10 mL/min.

The at least one sealing member 98 is configured to direct the flow of air from the second opening 20 to the air flow restrictor 21 through the porous plug 96 in an airtight manner. Advantageously, the at least one sealing member is an O-ring seal, a lip seal, a compression seal, etc. For instance, the regulation system 22 comprises two sealing members 98. A first sealing member 98 is advantageously arranged radially between the screw cap 92 and the rod 40. A second sealing member 98 is advantageously arranged radially around the porous plug 96, between the porous plug 96 and the screw cap 92.

### Description of Other Embodiments of the Invention

It will be apparent to those skilled in the art that other embodiments may be carried out in various ways by combining the previous embodiments.

For example, the first embodiment of the device 10 may be implemented without position-holding means 28 and/or positioners 39.

For example, the device 10 according to the third, fifth or the sixth embodiment may be implemented with a position-holding means 28 and/or positioners 39. For example, the flow restrictor 21 and the regulation system 22 may be implemented as in the third embodiment.

As another example, the fifth embodiment of the device 10 may be implemented with the regulation system 22 described in the third embodiment.

As another example, the first, second, third, fourth, fifth and sixth embodiment of the device 10 may be implemented with the regulation system 22 of the seventh embodiment.

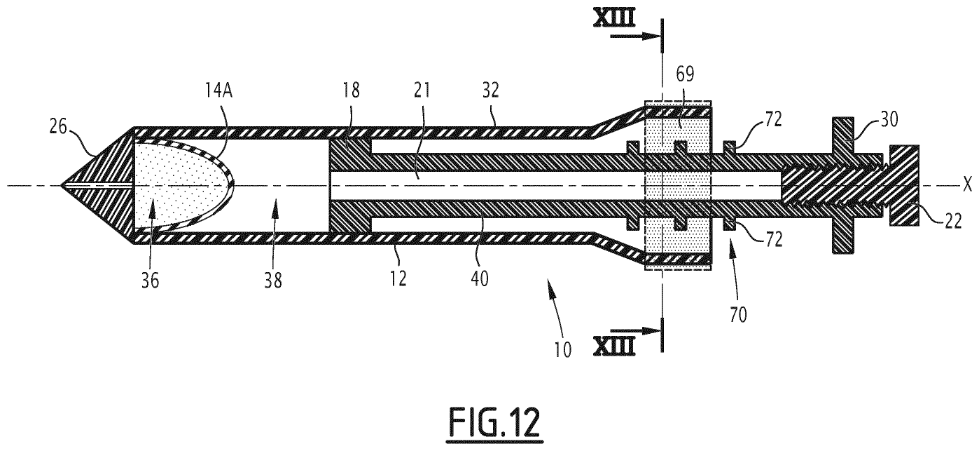

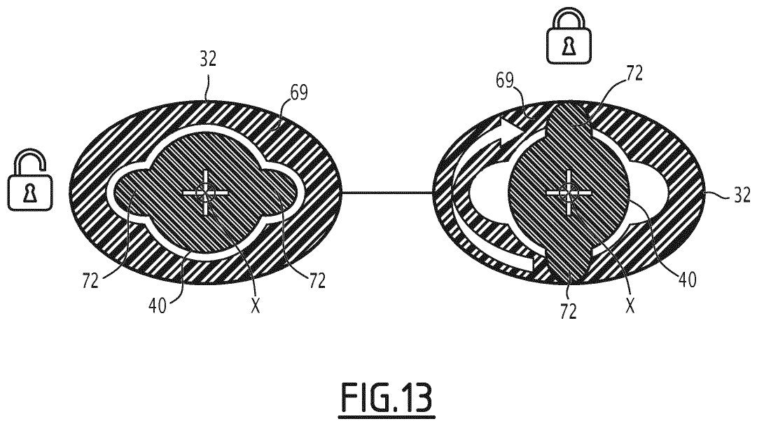

As a variant to each of the disclosed embodiments and as shown on FIGS. 12 and 13, the position-holding means 28 are configured to keep the piston 18 in position along the longitudinal axis X. For example, the position-holding means 28 comprise several groups 70 of stops 72 protruding radially from the rod 40 towards the barrel 12, in particular towards the internal surface 34 of the barrel 12.

For example, each group 70 of stops 72 is able to cooperate with the stop 69 protruding from the internal surface 34 of the barrel 12 inside the internal volume to keep the piston 18 in position along the longitudinal axis X. For instance, the stops 72 and the rod 40 are made-up in one piece. For example, each group 70 of stops comprises two stops 72 that are arranged symmetrically with reference to the longitudinal axis X. For example, the groups 70 of stops 72 are arranged spaced from one another along the longitudinal axis X. For example, two adjacent groups 70 of stops 72 are spaced apart from one another by a distance which is substantially equal to the thickness of the stop 69, said thickness being measured along the longitudinal axis X.

For example, the rod 40 is mounted rotatable around the longitudinal axis X between an unlocked configuration (shown on the left side of FIG. 13) and a locked configuration (shown on the right side of FIG. 13). In the unlocked configuration, the stops 72 protruding radially from the rod 40 towards the internal surface 34 of the barrel 12 are away from the stops 69 protruding from the internal surface 34 of the barrel 12 inside the internal volume. In the locked configuration, the stops 72 protruding radially from the rod 40 towards the internal surface 34 of the barrel 12 are applied against the stops 69 protruding from the internal surface 34 of the barrel 12 inside the internal volume.

For instance, as shown on FIG. 13, the stop 69 protruding from the internal surface 34 of the barrel 12 inside the internal volume has a cross-sectional shape (relative to the longitudinal axis X) which is substantially complementary to the cross-sectional shape of the ensemble comprising the rod 40 and a group 70 of stops 72 (relative to the longitudinal axis X) when the rod 40 is in the unlocked configuration. Thus, in the unlocked configuration, the piston 18 can move along the longitudinal axis X. In the locked configuration, the cross-sectional shape of the ensemble comprising the rod 40 and a group 70 of stops 72 (relative to the longitudinal axis X) and the cross-sectional shape of the stop 69 (relative to the longitudinal axis X) overlap. The stops 72 and 69 cooperate so that the piston 18 is kept in position along the longitudinal axis X. For example, in the locked configuration, the stops 72 and 69 are in abutment with each other. In a variant, in the locked configuration, the stops 72 and 69 are in friction cooperation.

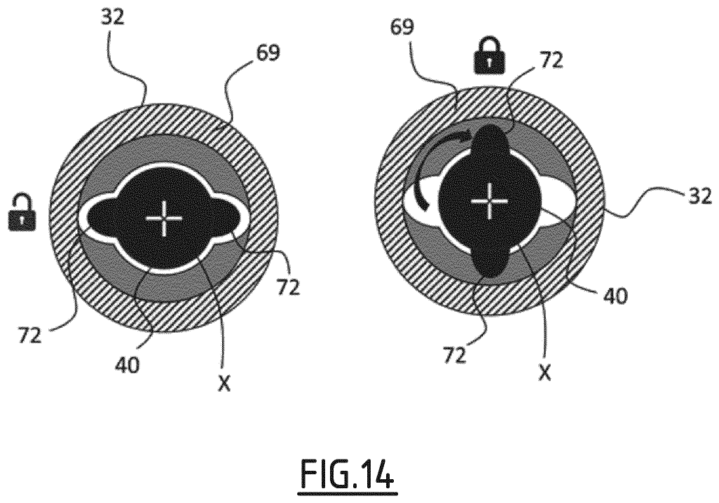

Advantageously, as shown on FIG. 14, the stop 69 comprises:
an upper abutment surface (not shown) extending from the internal surface 34 of the barrel 12 in a plane substantially perpendicular to the longitudinal axis X;
a lower abutment surface (not shown) extending from the internal surface 34 of the barrel 12 in a plane substantially perpendicular to the longitudinal axis X;
a circular hollow space presenting a diameter greater than or equal to the radial distance between distal ends of the stops 72 of a same group 70 and presenting a dimension taken along the longitudinal axis X which is substantially greater than or equal to a thickness of the stops 72 measured along the longitudinal axis X.

Thanks to these features, the stops 72 can slide within the circular hollow space in the locked configuration to prevent movement of the piston 40 in either directions along the longitudinal axis X.

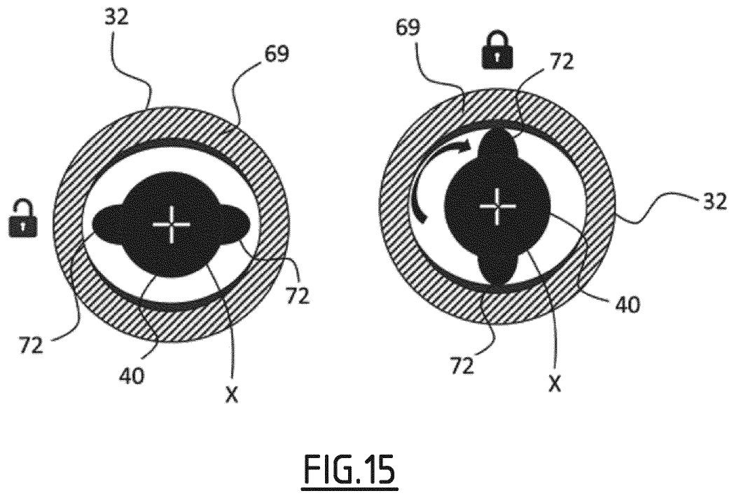

As a variant, as shown on FIG. 15, the stop 69 comprises an oval hollow space. In the locked configuration, the stops 72 are immobilized by friction cooperation with the stop 69.

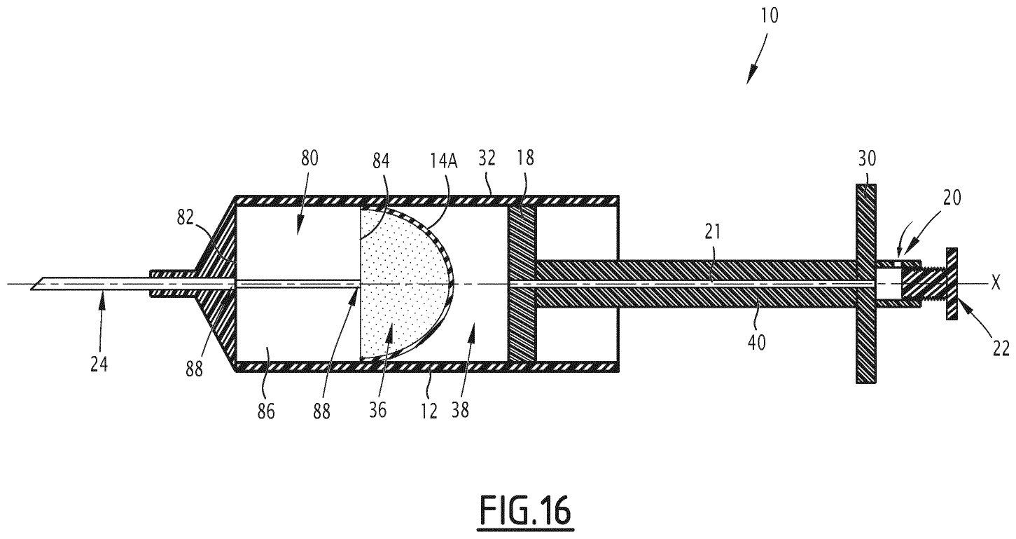

As a variant to each of the disclosed embodiments and as shown on FIG. 16, the elastic system 14 comprises a support 80. Advantageously, the support 80 is fixedly mounted into the internal volume of the barrel 12, in particular within the solution chamber 36 of said internal volume. In this case, the solution chamber 36 is delimited by the barrel 12, the elastic membrane 14A, the support 80 and, if applicable, the cap 26. For example, the support 80 is mounted onto the internal surface 34 of the barrel 12, in particular by friction cooperation between the support 80 and the internal surface 34. In a variant, the support 80 is mounted onto the cap 26. For instance, the support 80 comprises a first transversal wall 82, a second transversal wall 84 and a cylindrical wall 86 which extends along an axis substantially parallel to the longitudinal axis X between the first and second transversal walls 82, 84.

For instance, the elastic membrane 14A is mounted onto the support 80, in particular onto the second transversal wall 84 so as to wholly cover the second transversal wall 84. Advantageously, the elastic membrane 14A is preformed so that its shape is substantially complementary to the shape of the support 80, in particular to the shape of the second transversal wall 82, in the rest configuration.

Advantageously, the elastic membrane 14A is pre-tensioned so that the elastic membrane 14A is firmly applied against the support 80 even in the rest configuration. Thanks to this feature, the volume of the solution chamber 36 is substantially null in the rest configuration. For instance, the first transversal wall 82 and the second transversal wall 84 each comprise a passage hole 88 and the cylindrical wall 86 defines a hollow passage which allow the liquid solution to flow through the support 80 and reach the solution chamber 36.

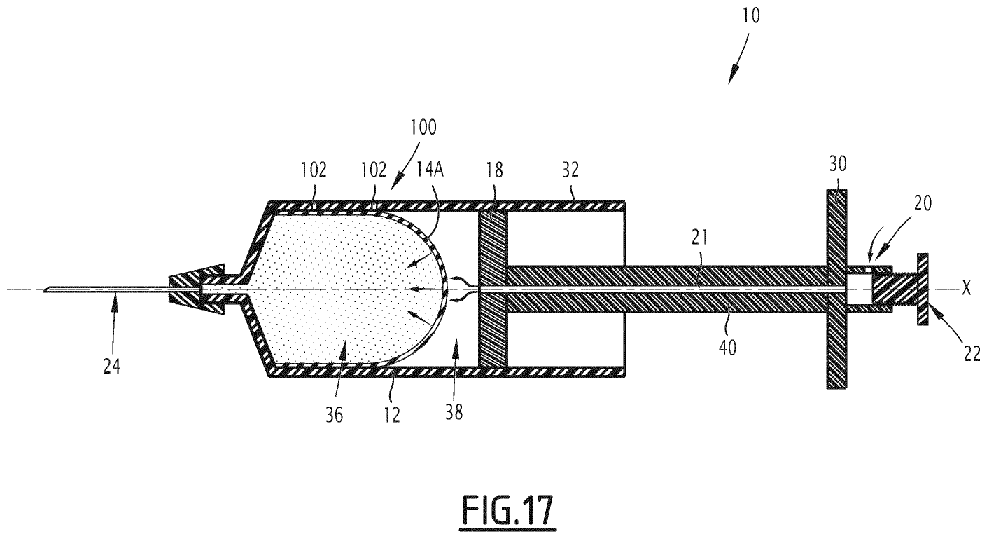

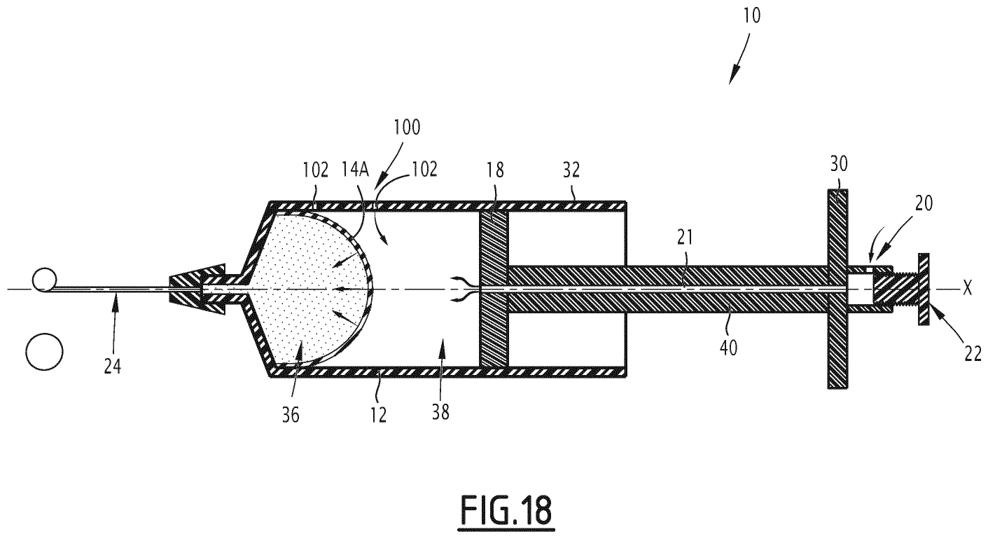

As a variant to each of the disclosed embodiments and as shown on FIGS. 17 and 18, the device comprises an airflow adjustment system 100. The airflow adjustment system 100 comprises at least one through-hole 102 extending in the barrel 12, in particular extending from the external surface 32 to the internal surface 34 of the barrel 12.

Advantageously, the airflow adjustment system 100 further comprises, for each through-hole 102, a filtering member (non-illustrated) covering said through-hole 102, and configured for filtering the air flowing through said through-hole 102. For instance, the filtering member is a porous track-etched membrane.

Advantageously, the airflow adjustment system 100 further comprises a plugging element (non-illustrated) which can be arranged onto the external surface 32 of the barrel 12, to cover the at least one through-hole 102 and prevent air from flowing through said at least one through-hole 102. For example, the plugging element is a self-adhesive element which can be adhered onto the at least one through-hole 102.

The at least one through-hole 102 is able to put in fluidic communication the internal volume of the barrel 12, in particular the depressurized chamber 38, with the ambient air. As shown on FIGS. 17 and 18, the at least one through-hole 102 is arranged so as to be plugged by elastic system, in particular by the elastic membrane 14A, when the piston 18 is in a pulled configuration and opened, the elastic membrane 14A being away from the at least one through-hole 102, when the piston is in the rest configuration.

As shown on the example of FIGS. 17 and 18, the air flow adjustment system 100 comprises a plurality of through holes 102 (here two through-holes) spaced apart from one another along the longitudinal axis X. These features make it possible to adjust the flow of air which flows within the depressurized chamber 38 as the piston 18 moves towards the rest configuration. Also, these features make it possible to maintain a linearity of the ejection of the solution out of the solution chamber.

1. A device for a controlled ejection of a liquid solution comprising:
a barrel defining an internal volume;
an elastic system separating the internal volume into a solution chamber and a depressurized chamber;
a first opening configured to allow the inlet and the outlet of said solution into the solution chamber;
a piston able to be moved inside the depressurized chamber from a rest configuration to a pulled configuration in order to suck said solution into the solution chamber through the first opening by the deformation of the elastic system creating a vacuum into the solution chamber, the elastic system following the movement of the piston, the elastic system having elastic forces, which tends it to come back in a rest configuration;
a second opening communicating with the depressurized chamber via a flow restrictor, and
a regulation system able to close the second opening to make the depressurized chamber airtight when the piston is sucking the solution and able to open the second opening to allow an airflow to flow inside the depressurized chamber via the flow restrictor to release the elastic system and to eject the solution out of the solution chamber through the first opening.

2. The device according to claim 1, wherein the elastic system is an elastic membrane fixed relatively to the barrel.

3. The device according to claim 1, wherein the elastic system is a mobile shutter in the internal volume of the barrel, the mobile shutter being connected to a spring arranged inside the depressurized chamber or inside the solution chamber.

4. The device according to claim 1, wherein the device comprises a cap removably cooperating with the barrel and defining the first opening.

5. The device according to claim 1, wherein the device comprises position-holding means configured to keep the piston in the pulled configuration.

6. The device according to claim 5, wherein the position-holding means are a stop protruding from the barrel inside the internal volume, the stop being able to cooperate with positioners to maintain the piston in the pulled configuration.

7. The device according to claim 1, wherein the device comprises a handle for moving the piston from the rest configuration to the pulled configuration.

8. The device according to claim 1, wherein the regulation system comprises a screw able to move in a matching hole from a screwed-in configuration blocking the fluidic communication between the second opening and the flow restrictor, to an unscrewed configuration enabling the fluidic communication between the second opening and the flow restrictor.

9. The device according to claim 1, wherein the flow restrictor is a capillary system extending between the second opening and the depressurized chamber.

10. The device according to claim 9, wherein the capillary system comprises at least one capillary channel extending between the second opening and the depressurized chamber.

11. The device according to claim 8, wherein the screw comprises externals threads and the matching hole comprises internal threads, the capillary system being a spiral capillary channel defined between the external threads and the internal threads.

12. The device according to claim 1, wherein the flow restrictor is a porous track-etched membrane.

13. The device according to claim 1, wherein the piston is able to move from the rest configuration to the pulled configuration through a translation movement, without rotation.

14. The device according to claim 1, wherein the device comprises a screw plunger cooperating with the piston, the piston being able to move from the rest configuration to the pulled configuration through a translation and rotation movement.

15. The device according to claim 1, further comprising a rod, the rod comprising the flow restrictor, the piston being connected to the rod.

16. The device according to claim 15, wherein the regulation system comprises:
a screw cap mounted movable onto the rod between a fully screwed on configuration and a plurality of unscrewed configurations, the second opening being defined by the screw cap; and
a porous plug arranged between the second opening and the flow restrictor;
wherein the screw cap pushes the porous plug against the rod so that the porous plug is compressed to make the depressurized chamber airtight when the screw cap is in the fully screwed on configuration,
wherein the screw cap enables the porous plug to expand, allowing air to flow inside the depressurized chamber via the flow restrictor to release the elastic membrane when the screw cap is in an unscrewed configuration.

17. The device according to claim 16, wherein the regulation system further comprises at least one sealing member, the at least one sealing member being configured to direct the flow of air from the second opening to the flow restrictor through the porous plug in an airtight manner.

18. The device according to claim 15, wherein the position-holding means are configured to keep the piston in position along the longitudinal axis,
the position-holding means comprising several groups of stops protruding radially from the rod towards the barrel,
the position-holding means comprising a stop protruding from the barrel inside the internal volume,
the rod being mounted rotatable around the longitudinal axis between an unlocked configuration and a locked configuration,
the stops protruding radially from the rod being away from the stops protruding from the barrel when the rod is in the unlocked configuration,
the stops protruding radially from the rod and the stops protruding from the barrel cooperating so that the piston is kept in position along the longitudinal axis when the rod is in the locked configuration.

19. The device according to claim 1, wherein the elastic system is an elastic membrane fixed relatively to the barrel, and wherein the elastic system comprises a support, the support being fixedly mounted into the internal volume of the barrel, the elastic membrane being mounted onto the support.

20. The device according to claim 1, further comprising an airflow adjustment system comprising at least one through-hole extending in the barrel, the at least one through-hole being able to put in fluidic communication the depressurized chamber with ambient air, the at least one through-hole being arranged so as to be plugged by the elastic system when the piston is in a pulled configuration and opened, when the piston is in the rest configuration.

21. A method of loading a liquid solution into a device, the method comprising:
providing a reservoir containing the solution,
providing the device according to claim 1, the piston being in the rest configuration and the regulation system closing the second opening,
placing the first opening in fluidic contact with solution contained in the reservoir,
moving the piston from the rest configuration to the pulled configuration and sucking said solution into the solution chamber.
